(19) United States
(12) Patent Application Publication
(10) Pub. No.: US 2006/0030749 A1
(43) Pub. Date: Feb. 9, 2006

(54) Sexual vibrating ring

(76) Inventors: Cheng-Hsiung Hung, Taipei City (TW); Wen-Shui Chuo, Taipei City (TW)

Correspondence Address: Cheng-Hsiung Hung & Wen-Shui Chuo, P.O. Box No. 6-57, Junghe, Taipei 235, TW

(21) Appl. No.: 11/126,189

(22) Filed: May 11, 2005

(30) Foreign Application Priority Data
Aug. 3, 2004 (CN) 2004200722353

(51) Int. Cl. A61F 5/00

(52) U.S. Cl. 600/38

(57) ABSTRACT

The present invention provides a sexual vibrating ring, comprising a belt-like O-ring and a flat containg portion. The belt-like O-ring is a ring made of a soft rubber material and the flat containg portion connects the ring, in addition, a vibrating device is placed inside the flat containg portion. A male can place the sexual vibrating ring over his sex and switch on the vibrating device inside the flat containg portion to achieve the objective of stimulating a female's vagina interior during sexual intercourse.

### BACKGROUND OF THE INVENTION

(a) Field of the Invention This invention relates to a device applied during sexual relations, in particular, to a ring with vibrating function to be placed over a male's sex.

(b) Description of the Prior Art

The sexual relations between a man and a woman in human evaluation plays a role in having children to carry on the family; in addition, more and more people take account of their sensual pleasures during sexual activity. Therefore, there has been various auxiliary devices on the market for sexual partners to enjoy mutual satisfying pleasures during sexual relations.

The “Vibrating Condom” of U.S. Pat. No. 5,377,692 disclose a vibrating condom or device having an inflatable vibrating region, which contacts the clitoris or vaginal walls and is activated by an electrical power unit for stimulating the most sensitive spot on a female partner's sex. Nevertheless, said patent design is not convenient for use as the embodiments of said patent required a tube to externally connect said electrical power unit, thereby increasing the difficulty in using a condom.

Another conventional known “vibrating condom” on the market is designed to be placed over the root of a male's penis for repeatedly stimulating a female partner's clitoris during sexual activity, so as to increase the sensations felt by his sexual partner, who can further reach sexual excitement and orgasm. Said vibrating condom applies a cylinder vibrating motor with a bigger volume size to cause a certain stimulation effect on clitoris of a female partner during sexual relation.

### SUMMARY OF THE INVENTION

In 1950, Ernest Grafenberg found that a spot, i.e., the G-point, on the front wall of a female's vagina can be directly stimulated by various ways to enable the female to have an orgasm during a sexual relation. Subsequently, some obstetrics and gynecology specialists conducted many experiments and researches on the G-points of females. Generally, the G-point is about 2 cm in diameter and located at the vagina front wall about half way between the pubic bone and the cervix at the positions between eleven and one o'clock about 2 to 3 cm depth from the vagina entrance; however, the size, the spot or the shape of each female's G-point differs, in addition, while a female's G-point is stimulated, the spot where the G-point is would be a slight swelling.

Currently, existing similar products fail to really stimulate a female's vagina wall interior during sexual intercourse, moreover, the U.S. Pat. No. 5,377,692 showing a “Vibrating Condom” is not convenient to be used. In view of the foregoing, the invention provides a sexual vibrating ring for effectively stimulating a female's vagina interior during sexual activity.

The sexual vibrating ring of the invention comprises a belt-like O-ring and a flat containg portion, wherein the O-ring is made of a soft rubber material and the flat containg portion connects the O-ring and enables a vibrating device to be placed therein, so that a male partner is able to place the sexual vibrating ring over his sexual organ and activate the vibrating device in the flat containg portion to stimulate the G-point of a female partner's vagina interior during sexual relation. Therefore, his sexual partner can easily attain orgasm during sexual relation and the male partner can simultaneously decrease his physical strength consumption; moreover, the vibrating device stimulates the penis surface to enable the male to feel the vibrating excitement during sexual activity.

To enable a further understanding of the structural features and contents of the invention, the brief description of the drawings below is followed by the detailed description of embodiment.

### BRIEF DESCRIPTION OF THE DRAWINGS

FIG. 1 is a front view of a sexual vibrating ring according to the invention.

FIG. 2 is a side view of the sexual vibrating ring according to the invention.

FIG. 3 is a top view of the sexual vibrating ring according to the invention.

FIG. 4 is a schematic diagram showing a vibrating device of the sexual vibrating ring according to the invention.

FIG. 5 is a perspective view illustrating the sexual vibrating ring in an embodiment according to the invention.

### DETAILED DESCRIPTION OF THE PREFERRED EMBODIMENTS

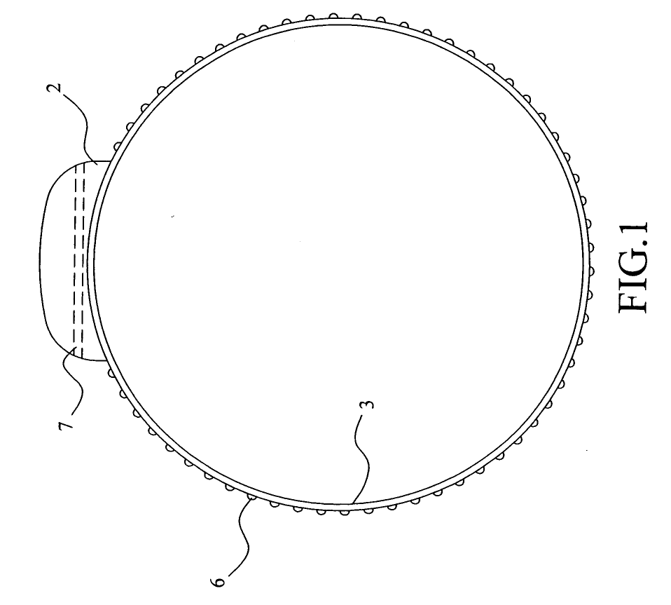

FIG. 1 shows a front view of a sexual vibrating ring of the invention, which is used to stimulate the vagina wall of a female's sex during sexual intercourse. A sexual vibrating ring 1 is formed by injecting soft non toxic rubber into one piece. The exterior of the sexual vibrating ring 1 is an elastic belt-like O-ring 3 with 0.2 mm in thickness and 6 mm in width and the inward part of the belt-like O-ring 3 is smooth to enable the O-ring 3 to be attached to a male's penis. In addition, the invention enables raised dots 6 with the dimension of 2 mm in height and 2 mm in width to be arranged on the exterior of the belt-like O-ring 3, the interval between every two raised dots is 6 mm. Consequently, said design of the present invention can increase the stimulation of the vagina during sexual relation.

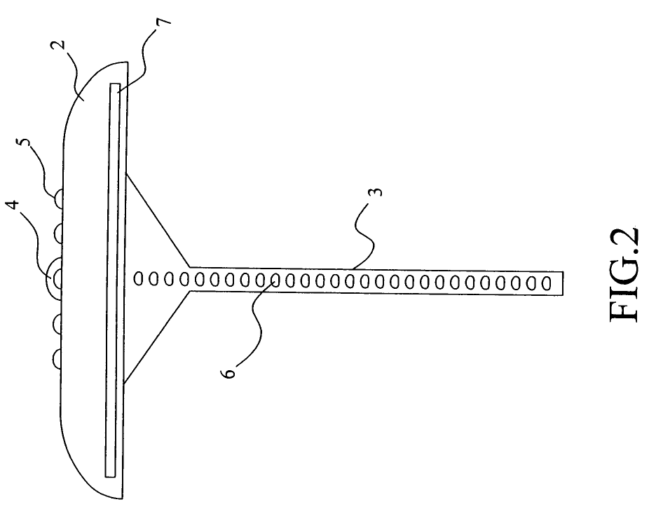

The flat containg portion 2 connects the belt-like O-ring 3, both are formed in one piece, in addition, the flat containg portion 2 is a hollow structure with mouths 7 on both ends, thereby a vibrating device is able to be placed therein and fixed inside the flat containg portion 2. The vibrating device is a flat vibrating device 8 to be placed inside an intermediate layer of the flat containg portion 2. The flat containg portion 2 is formed as an elliptic flat structure, the material thickness of which is about 1 mm dimension, in addition, the elliptic flat containg portion 2 is about 2.6 cm in long-diameter and 1.6 cm in short-diameter, the portion of the flat containg portion 2 in short-diameter and the belt-like O-ring 3 are formed in one piece. The flat containg portion 2 is a double hypotenuse design for strengthening the connection between the flat containg portion 2 and the belt-like O-ring 3. Said design can be referred to the side view of the sexual vibrating ring of the invention in FIG. 2.

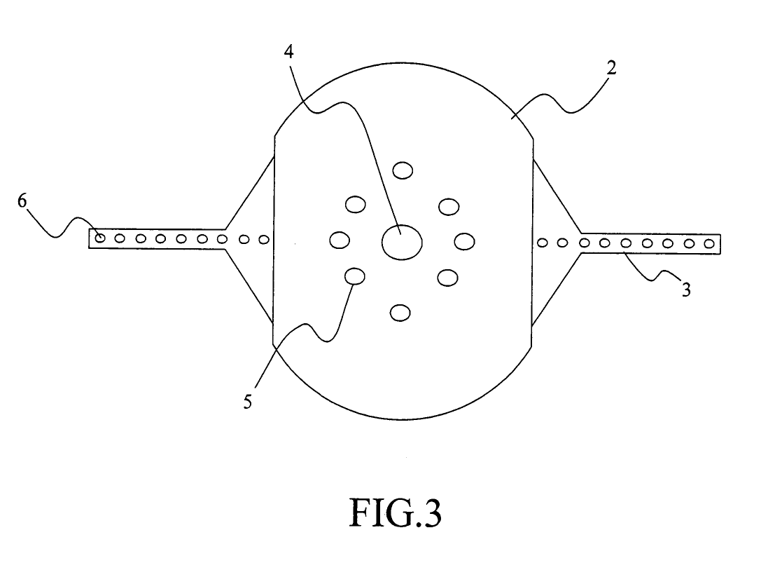

FIG. 3 shows a top view of the sexual vibrating ring of the invention, wherein the interior of the flat containg portion 2 is a smooth glossy surface, in order to closely attach to a male's sex (penis). Moreover, the exterior of the flat containg portion 2 has at least one elliptic soft raised dot 4 arranged on the top, the dimension of the elliptic soft raised dot 4 is 1 cm in length, 5 mm in width and 3 mm in height, in addition, the periphery of the elliptic soft raised dot 4 allows at least one raised dot 5 to be placed, in order to increase the stimulation of a female's vagina wall (the G-point) during sexual relation.

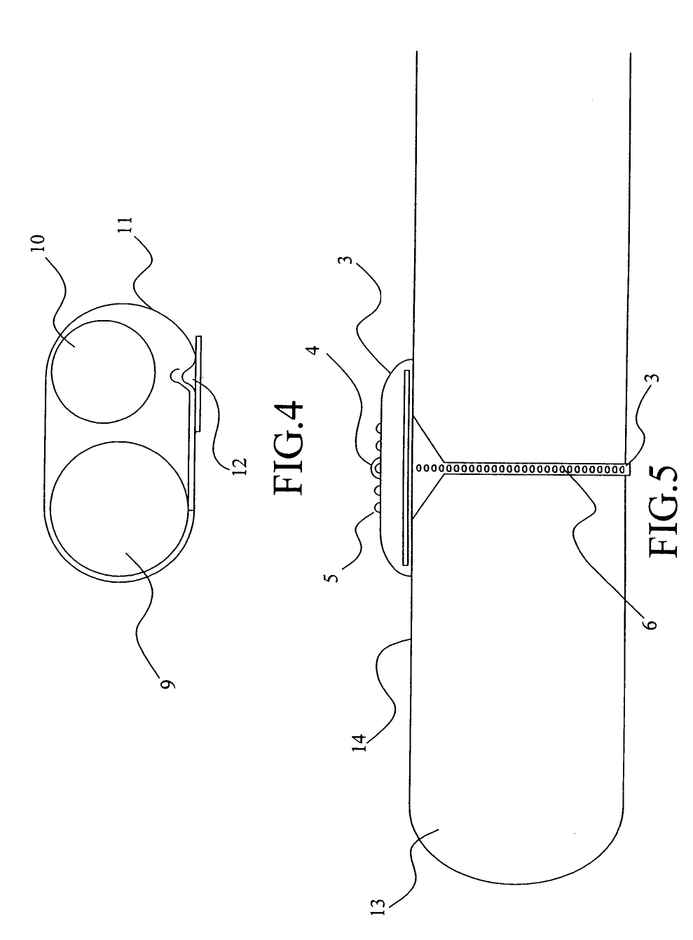

FIG. 4 is a schematic diagram showing the vibrating device of the sexual vibrating ring of the invention, wherin a plastic shell 11 is made of non toxic plastic material for placing a vibrating device therein. The shell is elliptic with 0.5 mm in thickness and the dimension is 2.3 cm in long diameter, 1.3 cm in short diameter and 4 mm in height. The plastic shell 11 has a flat vibrating motor 9 and a battery 10 placed therein, in addition, a power switch 12 is installed on the plastic shell 11 for powering on and off the flat vibrating motor 9.

The plastic shell 11 comprises an upper shell portion and a lower shell portion. First, the flat vibrating motor 9 and the battery 10 are both placed into the lower shell portion; subsequently, the upper shell portion and the lower shell portion are integrated together, therefore, the flat vibrating device 8 of the invention is completely assembled; consequently, the flat vibrating device 8 is placed into the flat containg portion 2, thereby the sexual vibrating ring of the invention 1 is completely assembled.

FIG. 5 is a perspective view illustrating the sexual vibrating ring in an embodiment according to the invention. Prior to sexual relations, a male places the sexual vibrating ring 1 over any spot of his sex 14 at the posterior portion of his balanus 13. The sexual vibrating ring 1 is made of a soft non-toxic rubber meterial with the elastic, soft and perfectly fit characteristics; therefore, said sexual vibrating ring 1 can be placed and closely attached to any portion of a male's penis without making him feel too tight. Moreover, the invention provides said sexual vibrating ring 1 for a male to wear at non-specific position of his sex, so that he can place the sexual vibrating ring 1 at any position or by any angle on his penis periphery and flexibly adjust or move the sexual vibrating ring 1, depending on the position (the G-point) on his female partner's vagina wall. After the sexual vibrating ring 1 is completely worn, he then switches on the power switch 12 for increasing the pleasures by applying the invention during sexual activity.

The vibration effect of the flat vibrating motor 9 influences respective raised dots 4, 5 and 6 on top of the flat containg portion 2 during sexual relation, so as to stimulate the sensitive position (the G-point) on a female's vagina wall enabling her to quickly reach orgasm, in addition, enabling her sexual partner to feel the vibrating stimulation on his penis owing to the vibration of the vibrating device 8. Consequently, the invention enables both female and male partners to increase greater mutual pleasures during sexual intercourse, settles the inability of a female to attain reach orgasm quickly by the vibrating stimulation of the invention to shorten the time of reaching orgasm a female, as well as to reduce the physical strength of a male during sexual activity.

Another advantage of the invention is to indirectly increase about 0.6 cm in diameter of a male penis after a male wears the invention for sexual relation, so that insufficient rubbing stimulation of a male's sex to a female's vagina wall during sexual intercourse can be improved; moreover, a male can wear the invention prior to a condom use over his sex for sexual relation the functions and effect of the invention will not be affected. The characteristics and advantages of the invention covered by this document have been set forth in the foregoing description. It is of course to be understood, however, that this disclosure is, in many respects, only illustrative. Changes may be made without exceeding the scope and the spirit of the invention. The scope of the invention is, of course, defined in the language in which the appended claims are expressed.

What is claimed is:

1. A sexual vibrating ring is placed over any spot of the sex at the posterior portion of balanus of a male for causing the vibration effect to stimulate a female's vagina interior during sexual intercourse, comprising:
a belt-like O-ring, an elastic ring for being placed over any spot of the sex at the posterior portion of the gaIns penis of a male;
a flat containg portion, a flat and a hollow structure connecting the belt-like O-ring;
a vibrating device, positioned inside the hollow structure for generating a vibrating source, further comprising:
a vibrating motor, a flat vibrating motor for generating the vibrating source;
a battery, connecting to the vibrating motor for providing required electricity for the vibrating motor; and
a power switch for controlling the status of the vibrating motor.

2. The sexual vibrating ring of claim 1, wherein the belt-like O-ring and the flat containg portion allow at least one raised dot thereon for increasing the vibrating stimulation of the sexual vibrating ring.
